(19) United States
(12) Patent Application Publication
(10) Pub. No.: US 2020/0370050 A1
(43) Pub. Date: Nov. 26, 2020

(54) CELL PROLIFERATION INHIBITOR AND CANCER TREATMENT OR PREVENTION PHARMACEUTICAL COMPOSITION INCLUDING CELL PROLIFERATION INHIBITOR

(71) Applicant: Yoshiro NIITSU, Hokkaido (JP)

(72) Inventor: Yoshiro NIITSU, Hokkaido (JP)

(21) Appl. No.: 16/772,578

(22) PCT Filed: Dec. 12, 2018
(86) PCT No.: PCT/JP2018/045627

§ 371 Date: Aug. 13, 2020

(30) Foreign Application Priority Data
Dec. 15, 2017 (JP) 2017-240652

(51) Int. Cl. C12N 15/113; A61K 38/45; A61P 35/00

(52) U.S. Cl. CPC C12N 15/1137; A61P 35/00; A61K 38/45

(57) ABSTRACT

The present invention provides a drug that makes it possible to effectively inhibit cancer cell growth. In particular, the present invention provides: a cell growth inhibitor for cancers in which the RAS/RAF/MEK/ERK signaling cascade has been activated, the cell growth inhibitor combining a drug that inhibits GSTP1 and a drug that inhibits the RAS/RAF/MEK/ERK signaling cascade; and a cell growth inhibitor for cancers in which the RAS/RAF/MEK/ERK signaling cascade has been activated, the cell growth inhibitor including a drug that inhibits interaction between GSTP1 and CRAF.

### TECHNICAL FIELD

The present invention relates to a cell growth inhibitor, specifically a cell growth inhibitor related to inhibition of GSTP1, and to a pharmaceutical composition, and so on, for treating or preventing cancer, which include the cell growth inhibitor.

### BACKGROUND ART

Cancer is one of the most important and troublesome diseases faced by mankind, and extensive research efforts have been made to treat it. Cancer is a disease in which cells grow uncontrolled due to mutations in genes or epigenetic abnormalities. Numerous genetic abnormalities in cancer have already been reported (see, for example, Patent Document 1), many of which are thought to have some association with signal transduction related to cell growth, differentiation, and survival. Such genetic abnormalities may also result in abnormalities in signal transduction in a cell composed of normal molecules, which may lead to activation or inactivation of a specific signaling cascade and ultimately result in abnormal cell growth. Initial cancer treatment focused primarily on inhibiting cell growth itself, but this treatment also inhibited the growth of physiologically normal growing cells, which was associated with side effects such as hair loss, digestive disorders, and myelosuppression. Therefore, to curb such side effects, cancer drugs are being developed based on new ideas, such as molecular targeted drugs that target cancer-specific genetic aberrations and abnormalities in signal transduction.

Glutathione-S-transferase (GST) is an enzyme that catalyzes glutathione conjugation, which adds glutathione to materials such as drugs. GST plays an important role in vivo, for example, in biosynthesis or drug metabolic degradation. GST is classified into multiple classes (e.g., α, μ, π, θ, etc.) based on primary structure homology and substrate specificity.

It has been pointed out that the expression of GSTP1 (also called glutathione S-transferase pi, GST-n) particularly increases in a variety of cancer cells, which may contribute to resistance to some anticancer drugs. In fact, it is known that drug resistance can be reduced when antisense DNAs to GSTP1 or GSTP1 inhibitors are allowed to act on cancer cell lines that overexpress GSTP1 and exhibit drug resistance (see Non-Patent Documents 2 to 4). It has also been reported that, when a siRNA against GSTP1 is allowed to act on an androgen-independent prostate cancer cell line overexpressing GSTP1, the growth is inhibited and apoptosis is enhanced (see Non-Patent Document 5).

As for GSTP1, Patent Document 1 discloses that using a GSTP1-inhibiting drug and an autophagy inhibitor such as 3-methyladenine as active ingredients can induce cancer cell apoptosis. Patent Document 2 discloses that simultaneous inhibition of GSTP1 expression and Akt expression results in inhibition of cell growth and induction of cell death and that the autophagy induced by the inhibition of GSTP1 expression is significantly reduced by the simultaneous inhibition of Akt expression. Patent Document 3 discloses an agent for inducing apoptosis, which includes a GSTP1-inhibiting drug and an RB1C1-inhibiting drug. Patent Document 4 discloses an agent for inducing death of cells having a mutation in the BRAF gene, which includes a GSTP1-inhibiting drug. Patent Document 5 discloses a cancer cell death-inducing agent including a GSTP1-inhibiting drug and a drug that inhibits a homeostasis maintenance-related protein that indicates synthetic lethality when inhibited together with GSTP1.

Unfortunately, the relationship between GSTP1, cell growth, and apoptosis, the molecular mechanism of GSTP1, and the role of GSTP1 in signal transduction in various cells remain largely unrevealed, and more research efforts are needed.
Patent Document 1: PCT International Publication No. WO2012/176282
Patent Document 2: PCT International Publication No. WO2014/098210
Patent Document 3: Japanese Unexamined Patent Application, Publication No. 2016-20337
Patent Document 4: Japanese Unexamined Patent Application, Publication No. 2016-204365
Patent Document 5: Japanese Unexamined Patent Application, Publication No. 2017-14185
Non-Patent Document 1: Futreal et al., Nat Rev Cancer. 2004; 4(3):177-83
Non-Patent Document 2: Takahashi and Niitsu, Gan To Kagaku Ryoho. 1994; 21(7):945-51
Non-Patent Document 3: Ban et al., Cancer Res. 1996; 56(15):3577-82
Non-Patent Document 4: Nakajima et al., J Pharmacol Exp Ther. 2003; 306(3):861-9
Non-Patent Document 5: Hokaiwado et al., Carcinogenesis. 2008; 29(6):1134-8

### DISCLOSURE OF THE INVENTION

### Problems to be Solved by the Invention

It is an object of the present invention to provide a pharmaceutical capable of effectively inhibiting cancer cell growth.

### Means for Solving the Problems

To solve the problems mentioned above, the present inventor has made extensive studies and, as a result, found that, when induced by activation of the RAS/RAF/MEK/ERK signaling cascade, GSTP1 binds to CRAF to enhance its activity. The inventor has also found that, when induced by activation of the RAS/RAF/MEK/ERK signaling cascade (the black arrow in FIG. 4), GSTP1 enhances the activity of CRAF, a component of the signaling cascade (GSTP1 autocrine loop; the white arrow in FIG. 4), independently of stimulation from upstream of the signaling cascade, so that the signaling cascade is aberrantly activated by both pathways.

Based on the findings, the inventor has revealed that combination use of a GSTP1-inhibiting drug (e.g., siRNA against GSTP1 gene) and a RAS/RAF/MEK/ERK signaling cascade-inhibiting drug (e.g., siRNA against KRAS gene) to inhibit both the GSTP1 autocrine loop and the RAS/RAF/MEK/ERK signaling cascade is more effective in inhibiting cancer cell growth than use of only one of these drugs. The inventor has further found that cancer cell growth can be inhibited by using a drug such as a CRAF decoy peptide to inhibit interaction between GSTP1 and CRAF at the junction between the RAS/RAF/MEK/ERK signaling cascade and the GSTP1 autocrine loop. Based on these findings, the inventor has completed the present invention.

Specifically, the present invention includes the following aspects directed to:

(1) A cell growth inhibitor against a cancer in which a RAS/RAF/MEK/ERK signaling cascade is activated, the cell growth inhibitor including a combination of a GSTP1-inhibiting drug and a RAS/RAF/MEK/ERK signaling cascade-inhibiting drug;
(2) A cell growth inhibitor against a cancer in which a RAS/RAF/MEK/ERK signaling cascade is activated, the cell growth inhibitor including a GSTP1-inhibiting drug to be administered in combination with a RAS/RAF/MEK/ERK signaling cascade-inhibiting drug;
(3) A cell growth inhibitor against a cancer in which a RAS/RAF/MEK/ERK signaling cascade is activated, the cell growth inhibitor including a RAS/RAF/MEK/ERK signaling cascade-inhibiting drug to be administered in combination with a GSTP1-inhibiting drug;
(4) The cell growth inhibitor according to any one of aspects (1) to (3), wherein the cancer in which a RAS/RAF/MEK/ERK signaling cascade is activated is a cancer having an activating mutation in RAS;
(5) The cell growth inhibitor according to any one of aspects (1) to (4), wherein the cancer is colon cancer;
(6) The cell growth inhibitor according to any one of aspects (1) to (5), wherein the RAS/RAF/MEK/ERK signaling cascade-inhibiting drug is a RAS-inhibiting drug;
(7) The cell growth inhibitor according to any one of aspects (1) to (6), wherein the GSTP1-inhibiting drug is a siRNA against GSTP1;
(8) The cell growth inhibitor according to any one of aspects (1) to (7), wherein the RAS/RAF/MEK/ERK signaling cascade-inhibiting drug is a siRNA against a component of the RAS/RAF/MEK/ERK signaling cascade;
(9) A cell growth inhibitor against a cancer in which a RAS/RAF/MEK/ERK signaling cascade is activated, the cell growth inhibitor including a drug that inhibits interaction between GSTP1 and CRAF;
(10) The cell growth inhibitor according to aspect (9), wherein the cancer in which a RAS/RAF/MEK/ERK signaling cascade is activated is a cancer having an activating mutation in RAS;
(11) The cell growth inhibitor according to aspect. (9) or (10), wherein the cancer is colon cancer;
(12) The cell growth inhibitor according to any one of aspects (9) to (11), wherein the drug that inhibits interaction between GSTP1 and CRAF is a CRAF decoy peptide or a vector that expresses the CRAF decoy peptide;
(13) The cell growth inhibitor according to aspect (12), wherein the CRAF decoy peptide is selected from the group consisting of
(a) a polypeptide having an amino acid sequence set forth in SEQ TD NO:9,
(b) a polypeptide having an amino acid deletion, substitution, or addition at one or multiple positions in the amino acid sequence set forth in SEQ ID NO: 9,
(c) a polypeptide having at least 90% sequence identity to the amino acid sequence set forth in SEQ ID No: 9, and
(d) a polypeptide having 1 to 50 amino acid residues added to an N- or C-terminus of the polypeptide defined in any one of (a) to (c);
(14) A pharmaceutical composition for treating or preventing a cancer in which a RAS/RAF/MEX/ERK signaling cascade is activated, the pharmaceutical composition including the cell growth inhibitor according to any one of aspects (1) to (13); and
(15) A kit for treating or preventing a cancer in which a RAS/RAF/MEK/ERK signaling cascade is activated, the kit including the cell growth inhibitor according to any one of aspects (1) to (13).

### Effects of the Invention

The description includes the contents disclosed in Japanese Patent Application No. 2017-240652 based on which the present application claims priority.

According to the present invention, there are provided pharmaceuticals capable of effectively inhibiting cancer cell growth.

### BRIEF DESCRIPTION OF THE DRAWINGS

FIG. 1 is a schematic diagram showing the structures of proteins expressed by plasmids used in coimmunoprecipitation experiments with GSTP1 shown in Example 1;

FIG. 2 is a photograph showing the results of coimmunoprecipitation experiments with GSTP1 shown in Example 1;

FIG. 3 is a photograph showing the results of an in vitro kinase assay for examining the effect of GSTP1 on CRAF activity shown in Example 2;

FIG. 4 is a schematic diagram showing the promotion of the RAS/RAF/MEK/ERK signaling cascade in KRAS mutation-positive cancer cells;

FIG. 5 is a graph showing the results of examining the effect of a CRAF protein fragment on cell growth shown in Example 3; and

FIG. 6 is a graph showing the results of examining the effect of double inhibition of GSTP1 and KRAS on cell growth shown in Example 4. The asterisks indicate P<0.01.

### PREFERRED MODE FOR CARRYING OUT THE INVENTION

Hereinafter, the present invention will be described in detail.

### 1. Combination Use of GSTP1-Inhibiting Drug and RAS/RAF/MEK/ERK Signaling Cascade-Inhibiting Drug to Inhibit Cell Growth

The present invention relates to a cell growth inhibitor including a combination of a GSTP1-inhibiting drug and a RAS/RAF/MEK/ERK signaling cascade-inhibiting drug. The present invention is based on the inventor's findings that, as shown in the examples below, combination use of a GSTP1-inhibiting drug and a RAS/RAF/MEK/ERK signaling cascade-inhibiting drug to inhibit both the GSTP1 autocrine loop and the RAS/RAF/MEK/ERK signaling cascade is more effective in inhibiting cancer cell growth than use of only one of these drugs.

As used herein, the term “GSTP1” (GSTP1 protein) refers to an enzyme, encoded by GSTP1 gene, that catalyzes glutathione conjugation. GSTP1 is present in a variety of animals, including humans, and its sequence information is also known. The GSTP1 sequence information is available from public databases such as the NCBI database.

Specific examples of GSTP1 include a human-derived GSTP1 (human GSTP1) protein having the amino acid sequence of 210 residues set forth in SEQ ID NO: 1 (NCBI Accession Number NP_000843.1). The term “GSTP1” also includes GSTP1 variants and GSTP1 orthologs of other biological species, which have activity functionally equivalent to that of GSTP1 set forth in SEQ ID NO: 1. GSTP1 has glutathione conjugation catalytic activity, and methods for measuring the activity are known to those skilled in the art. Specifically, the term “GSTP1” includes GSTP1 proteins having an amino acid deletion, substitution, or addition at one or multiple positions in the amino acid sequence set forth in SEQ ID NO: 1 or having at least 90%, 95%, 97%, 98%, or 99% sequence identity to the amino acid sequence set forth in SEQ ID NO: I.

As used herein, the term “multiple” as to the deletion, substitution, or addition of amino acids or bases refers to, for example, 2 to 20, 2 to 15, 2 to 10, 2 to 7, 2 to 5, 2 to 4, or 2 to 3. Amino acid substitution is preferably conservative amino acid substitution. The term “conservative amino acid substitution” refers to substitution between amino acids similar in nature such as charge, side chain, polarity, or aromaticity. Amino acids similar in nature may be classified into, for example, basic amino acids (arginine, lysine, histidine), acidic amino acids (aspartic acid, glutamic acid), uncharged polar amino acids (glycine, asparagine, glutamine, serine, threonine, cysteine, tyrosine), non-polar amino acids (leucine, isoleucine, alanine, valine, proline, phenylalanine, tryptophan, methionine), branched chain amino acids (leucine, valine, isoleucine), and aromatic amino acids (phenylalanine, tyrosine, tryptophan, histidine).

As used herein, the term “sequence identity” refers to the identity in base sequence between two nucleic acids or in amino acid sequence between two proteins. Sequence identity is determined by comparing two optimally aligned sequences over a region in the target sequence. The nucleic acids or proteins to be compared may have an addition or deletion (e.g., a gap) in the two optimally aligned sequences. Sequence identity may be calculated using a search system such as BLAST or FASTA.

The term “GSTP1 gene” refers to a gene encoding the GSTP1. Specific examples of the GSTP1 gene include a human GSTP1 gene encoding a human GSTP1 having the amino acid sequence set forth in SEQ ID NO: 1. More specifically, the GSTP1 gene may be a gene having the base sequence set forth in SEQ ID NO: 2 (NCBI Accession Number NM_000852.3). The term “GSTP1 gene” also includes GSTP1 genes encoding GSTP1 variants or GSTP1 orthologs of other biological species, which have activity functionally equivalent to that of the human GSTP1 having the amino acid sequence set forth in SEQ ID NO: 1. Specifically, the term “GSTP1 gene” includes GSTP1 genes having a base deletion, substitution, or addition at one or multiple positions in the base sequence set forth in SEQ ID NO: 2 or having at least 90%, 95%, 97%, 98%, or 99% sequence identity to the based sequence set forth in SEQ ID NO: 2.

In the present disclosure, examples of the “GSTP1-inhibiting drug” include, but are not limited to, drugs that inhibit the production and/or activity of GSTP1, and drugs that promote the decomposition and/or inactivation of GSTP1.

Examples of drugs that inhibit the production of GSTP1 include, but are not limited to, inhibitory nucleic acids against the GSTP1 gene, such as RNAi molecules, ribozymes, antisense nucleic acids, and DNA/RNA chimeric polynucleotides, and vectors that express them. Such inhibitory nucleic acids and vectors that express them are preferred because of their high specificity and low potential for side effects.

As used herein, the term “RNAi molecule” refers to any molecule that produces RNA interference, examples of which include, but are not limited to, siRNA (small interfering RNA), miRNA (micro RNA), shRNA (short hairpin RNA), ddRNA (DNA-directed RNA), piRNA (Piwi-interacting RNA), rasiRNA (repeat associated siRNA), and other double stranded RNAs, and modifications thereof. These RNAi molecules are commercially available or can be designed and made based on known sequence information, such as base sequence information set forth in SEQ ID NO: 2.

As used herein, the term “antisense nucleic acid” refers to an antisense oligonucleotide having a base sequence complementary to a transcript (sense strand) of a target gene. The antisense nucleic acid may include RNA, DNA, PNA (peptide nucleic acid), LNA (locked nucleic acid), or any complex thereof.

In the present disclosure, examples of the “DNA/RNA chimeric polynucleotide” include, but are not limited to, a double stranded polynucleotide including DNA and RNA which inhibit expression of a target gene, such as that disclosed JP-A-2003-219893.

In the present disclosure, examples of the “expression vector” that may be used include, but are not limited to, any known vectors such as plasmid vectors, phage vectors, phagemid vectors, cosmid vectors, and viral vectors. The vector preferably contains at least a promoter that enhances expression of the carrying nucleic acid, in which, preferably, the nucleic acid is operably linked to the promoter. The expression “a nucleic acid is operably linked to a promoter” means that the nucleic acid and the promoter are arranged such that the promoter can act to allow proper production of a protein encoded by the nucleic acid. The vector may be capable of being replicated in a host cell. The gene may be transcribed from the vector outside the nucleus of the host cell or inside the nucleus of the host cell (for example, by inserting the nucleic acid into the genome of the host cell).

Examples of drugs that inhibit the activity of GSTP1 include, but are not limited to, substances that bind to GSTP1, such as glutathione, glutathione analogs (e.g., those disclosed in WO 95/08563, WO 96/40205, WO 99/54346, or Nakajima et al., J Pharmacol Exp Ther. 2003; 306(3): 861-9), ketoprofen (Takahashi and Niitsu, Gan To Kagaku Ryoho. 1994; 21(7): 945-51), indomethacin (Hall et al., Cancer Res. 1989; 49 (22): 6265-8), ethacrynic acid, piriprost (Tew et al., Cancer Res. 1988; 48(13): 3622-5), anti-GSTP1 antibodies, and dominant negative mutants of GSTP1. These drugs are commercially available or can be produced as appropriate based on known techniques.

Whether GSTP1 is inhibited can be determined by determining whether the expression (expressed amount) and/or activity of GSTP1 in cells is inhibited as compared to a case where no drug is allowed to act to inhibit GSTP1.

The expression of GSTP1 may be assessed by any known methods, which include, but are not limited to, methods using anti-GSTP1 antibodies such as immunoprecipitation, EIA (enzyme immunoassay) (e.g., ELISA (enzyme-linked immunosorbent assay)), RTA (radioimmunoassay) (e.g., IRMA (immunoradiometric assay)), RAST (radioallergosorbent test), RIST (radioimmunosorbent test)), Western blotting, immunohistochemistry, immunocytochemistry or flow cytometry, or techniques using GSTP1 gene transcripts (e.g., mRNA) or splicing products, or using nucleic acids that specifically hybridize fragments thereof, such as various hybridization methods including Northern blotting or Southern blotting, or various PCR techniques (e.g., real-time RT-PCR).

The activity of GSTP1 may be assessed by analyzing the known activity of GSTP1, which includes, but is not limited to, the ability to bind to a protein such as CRAF (specifically phosphorylated CRAF) or EGFR (specifically phosphorylated EGFR), using any known method such as immunoprecipitation, Western blotting, mass spectrometry, pull-down assay, or surface plasmon resonance (SPR) technique.

As used herein, the term “signaling cascade” means signal transduction in which multiple signaling molecules transmit signals one after another. The term “RAS/RAF/MEK/ERK signaling cascade” refers to a signaling cascade involved in cell growth, cell differentiation, and so on and including RAS, RAF, MEK, and ERK as signaling molecules. When a ligand such as a growth factor binds to a G protein-coupled receptor or a tyrosine kinase-type receptor, RAS, a low molecular weight G protein, is activated, which then activates RAF (a type of MAPKKK) by phosphorylation. The activated RAF activates MEK (MAPK/ERK kinase, a type of MAP2K), and the activated MEK activates ERK (extracellular signal-regulated kinase, a type of MAPK). The activated ERK migrates to the nucleus and promotes the transcription of various types of mRNA to trigger cell growth.

Components of the RAS/RAF/MEK/ERK signaling cascade include RAS, RAF, MEK, and ERK.

As used herein, the term “RAS” (RAS protein) refers to a low molecular weight GTP binding protein encoded by the RAS gene. RAS is present in various animals, including humans, and its sequence information is also known. RAS sequence information is available from public databases such as the NCBI database. The term “RAS” includes KRAS, NRAS, and HRAS.

For example, specific examples of KRAS include a human-derived KRAS (human KRAS) protein having the amino acid sequence of 189 residues set forth in SEQ ID NO: 3 (NCBI Accession Number NP_203524.1). The term “KRAS” also includes KRAS variants and KRAS orthologs of other biological species, which have activity functionally equivalent to that of KRAS set forth in SEQ ID NO: 3. KRAS have GTP-hydrolyzing activity, and methods for measuring the activity are known to those skilled in the art. Specifically, the term “KRAS” includes KRAS proteins having an amino acid deletion, substitution, or addition at one or multiple positions in the amino acid sequence set forth in SEQ ID NO: 3 or having at least 90%, 95%, 97%, 98%, or 99% sequence identity to the amino acid sequence set forth in SEQ ID NO: 3.

The term “KRAS gene” refers to a gene encoding the KRAS. Specific examples of the KRAS gene include a human KRAS gene encoding a human KRAS having the amino acid sequence set forth in SEQ ID NO: 3. More specifically, the KRAS gene may be a gene having the base sequence set forth in SEQ ID NO: 4 (NCBI Accession Number NM_033360.3). The term “KRAS gene” also includes KRAS genes encoding KRAS variants or KRAS orthologs of other biological species, which have activity functionally equivalent to that of the human KRAS having the amino acid sequence set forth in SEQ ID NO: 3. Specifically, the term “KRAS gene” includes KRAS genes having a base deletion, substitution, or addition at one or multiple positions in the base sequence set forth in SEQ ID NO: 4 or having at least 90%, 95%, 97%, 98%, or 99% sequence identity to the base sequence set forth in SEQ ID NO: 4.

As used herein, the term “RAF” (RAF protein) refers to an enzyme, encoded by the RAF gene, that has kinase activity. RAF is present in various animals, including humans, and its sequence information is also known. RAF sequence information is available from public databases such as the NCBI database. The term “RAF” includes ARAF, BRAF, and CRAF (also called Raf-1).

For example, specific examples of CRAF include a human-derived CRAF (human CRAF) protein having the amino acid sequence of 648 residues set forth in SEQ ID NO: 5 (NCBI Accession Number NP_001341619.1) or having the amino acid sequence of 567 residues set forth in SEQ ID NO: 7 (NCBI Accession Number NP_001341620.1). The term “CRAF” also includes CRAF variants and CRAF orthologs of other biological species, which have activity functionally equivalent to that of CRAF set forth in SEQ ID NO: 5 or 7. Specifically, the term “CRAF” includes CRAF proteins having an amino acid deletion, substitution, or addition at one or multiple positions in the amino acid sequence set forth in SEQ ID NO: 5 or 7 or having at least 90%, 95%, 97%, 98%, or 99% sequence identity to the amino acid sequence set forth in SEQ ID NO: 5 or 7.

The term “CRAF gene” refers to a gene encoding the CRAF. Specific examples of the CRAF gene include a human CRAF gene encoding a human CRAF having the amino acid sequence set forth in SEQ ID NO: 5 or 7. More specifically, the CRAF gene may be a gene having the base sequence set forth in SEQ ID NO: 6 (NCBI Accession Number NM_001354690.1) or having the base sequence set forth in SEQ ID NO: 8 (NCBI Accession Number NM_001354691.1). The term “CRAF gene” also includes CRAF genes encoding CRAF variants or CRAF orthologs of other biological species, which have activity functionally equivalent to that of the human CRAF having the amino acid sequence set forth in SEQ ID NO: 5 or 7. Specifically, the term “CRAF gene” includes CRAF genes having a base deletion, substitution, or addition at one or multiple positions in the base sequence set forth in SEQ ID NO: 6 or 8 or having at least 90%, 95%, 97%, 98%, or 99% sequence identity to the base sequence set forth in SEQ ID NO: 6 or 8.

As used herein, the term “MEK” (MEK protein) refers to an enzyme, encoded by the MEK gene, that has kinase activity. MEK is present in various animals, including humans, and its sequence information is also known. MEK sequence information is available from public databases such as the NCBI database. The term “MEK” includes MEK1 and MEK2.

As used herein, the term “ERK” (ERK protein) refers to an enzyme, encoded by the ERK gene, that has kinase activity. ERK is present in various animals, including humans, and its sequence information is also known. ERK sequence information is available from public databases such as the NCB database. The term “ERK” includes ERK1 and ERK2.

In the present disclosure, examples of the “RAS/RAF/MEK/ERK signaling cascade-inhibiting drug” include, but are not limited to, drugs that inhibit the production and/or activity of a component(s) of the RAS/RAF/MEK/ERK signaling cascade, and drugs that promote the degradation and/or inactivation of a component(s) of the RAS/RAF/MEK/ERK signaling cascade.

Examples of drugs that inhibit the production of a component(s) of the RAS/RAF/MEK/ERK signaling cascade include, but are not limited to, inhibitory nucleic acids against genes encoding a component(s) of the RAS/RAF/MEK/ERK signaling cascade, such as RNAi molecules, ribozymes, antisense nucleic acids, and DNA/RNA chimeric polynucleotides, and vectors that express them. Such inhibitory nucleic acids and vectors that express them are preferred because of their high specificity and low potential for side effects.

Examples of drugs that inhibit the activity of a component(s) of the RAS/RAF/MEK/ERK signaling cascade include, but are not limited to, MEK inhibitors such as selumetinib and trametinib; BRAF inhibitors such as vemurafenib and PLX4720; ERK inhibitors; substances that bind to a component(s) of the RAS/RAF/MEK/ERK signaling cascade (e.g., antibodies that bind to a component(s) of the RAS/RAF/MEK/ERK signaling cascade); and dominant negative variants of a component(s) of the RAS/RAF/MEK/ERK signaling cascade. These drugs are commercially available or can be produced as appropriate based on known techniques.

In one embodiment, the RAS/RAF/MEK/ERK signaling cascade-inhibiting drug may be a RAS-inhibiting drug. The RAS-inhibiting drug may be a KRAS-inhibiting drug. The KRAS-inhibiting drug may be an inhibitory nucleic acid against the KRAS gene, such as an RNAi molecule.

A single drug that inhibits the RAS/RAF/MEK/ERK signaling cascade may be used, or two or more drugs that inhibit the RAS/RAF/MEK/ERK signaling cascade may be used (e.g., two or more drugs that inhibit different components of the RAS/RAF/MEK/ERK signaling cascade).

Whether the RAS/RAF/MEK/ERK signaling cascade is inhibited can be determined by determining whether the RAS/RAF/MEK/ERK signaling cascade is inhibited in cells as compared to a case where no drug is allowed to act to inhibit the RAS/RAF/MEK/ERK signaling cascade. As used herein, the term “signaling cascade-inhibiting” means not only the ability to induce the inactivation of the signaling cascade but also the ability to inhibit the activation of the signaling cascade. As a non-limiting example, whether the RAS/RAF/MEK/ERK signaling cascade is inhibited can be assessed by determining the expression (expressed amount) of a component(s) of the RAS/RAF/MEK/ERK signaling cascade or the amount of a phosphorylated component(s) of the RAS/RAF/MEK/ERK signaling cascade using any known technique (e.g., antibody-based techniques such as immunoprecipitation or Western blotting, or nucleic acid-based techniques such as various hybridization techniques such as Northern blotting or Southern blotting, or various PCR techniques).

The cell growth inhibitor according to the present invention may be used to treat a cancer in which the RAS/RAF/MEK/ERK signaling cascade is activated. As used herein, the expression “signaling cascade is activated” means not only induction of activation of the signaling cascade but also inhibition of inactivation of the signaling cascade.

As used herein, the expression “cancer in which the RAS/RAF/MEK/ERK signaling cascade is activated” may include cancers that have an activating mutation in a component(s) of the RAS/RAF/MEK/ERK signaling cascade, or cancers that are associated with an increase in the expression (expressed amount) of a component(s) of the RAS/RAF/MEK/ERK signaling cascade, cancers that are associated with an increase in the amount of a phosphorylated component(s) of the RAS/RAF/MEK/ERK signaling cascade, and cancers that are associated with activation of the signaling cascade by related factors other than the components of the RAS/RAF/MEK/ERK signaling cascade (e.g., activation of receptor tyrosine kinase). As used herein, the term “activating mutation” refers to a mutation that causes permanent activation of the function of a protein. In the present disclosure, “cancer having a mutation” may also be referred to as “mutation-positive cancer”.

Mutations in a component(s) of the RAS/RAF/MEK/ERK signaling cascade may be detected by any known techniques, examples of which include, but are not limited to, selective hybridization using nucleic acid probes specific for known mutated sequences, enzymatic mismatch cleavage, sequencing, and PCR-RFLP (polymerase chain reaction-restriction fragment length polymorphism).

In one embodiment, the cancer in which the RAS/RAF/MEK/ERK signaling cascade is activated may be a cancer having an activating mutation in RAS (e.g., KRAS). In the present disclosure, the cancer having an activating mutation in RAS may be a cancer having, in RAS, a mutation that inhibits endogenous GTPase or a mutation that increases the rate of guanine nucleotide exchange. Specific examples of such mutations include, but are not limited to, amino acid mutations at positions 12, 13 and/or 61 in human KRAS (for inhibiting endogenous GTPase) or amino acid mutations at positions 116 and/or 119 in human KRAS (for increasing the rate of guanine nucleotide exchange) (Bos, Cancer Res. 1989; 49(17): 4682-9, Levi et al., Cancer Res. 1991; 51(13): 3497-502). In one embodiment, therefore, the KRAS having an activating mutation may be a KRAS having an amino acid mutation at at least one of positions 12, 13, 61, 116, and 119 in the human KRAS. In one embodiment, the KRAS having an activating mutation has an amino acid mutation at position 13 in the human KRAS (e.g., an amino acid mutation at position 13 from glycine to aspartic acid).

In one embodiment, the cancer in which the RAS/RAF/MEK/ERK signaling cascade is activated may be a cancer that overexpresses GSTP1. In the present disclosure, the expression of GSTP1 may be detected using any known techniques including those mentioned above. Whether GSTP1 is overexpressed in test cells (e.g., cancer cells) may be assessed, for example, by comparing the level of expression of GSTP1 in the test cells with the level of expression of GSTP1 in normal cells of the same type. In this case, GSTP1 can be determined as being overexpressed if the level of expression of GSTP1 in the test cells exceeds that of GSTP1 in the normal cells of the same type.

In the present disclosure, examples of the cancer include, but are not limited to, sarcomas such as fibrosarcoma, malignant fibrous histiocytoma, liposarcoma, rhabdomyosarcoma, leiomyosarcoma, angiosarcoma, Kaposi's sarcoma, lymphangiosarcoma, synovial sarcoma, chondrosarcoma, and osteosarcoma; cancers such as brain tumor, head and neck cancer, breast cancer, lung cancer, esophageal cancer, gastric cancer, duodenal cancer, appendiceal cancer, colon cancer, rectal cancer, liver cancer, pancreatic cancer, gallbladder cancer, bile duct cancer, anal cancer, renal cancer, ureteral cancer, bladder cancer, prostate cancer, penile cancer, testicular cancer, uterine cancer, ovarian cancer, vulvar cancer, vaginal cancer, and skin cancer; and leukemia and malignant lymphoma. As used herein, the term “cancer” includes epithelial and non-epithelial malignancies. Cancers can be present at any site of the body, such as brain, head and neck, chest, limbs, lung, heart, thymus, esophagus, stomach, small intestine (duodenum, jejunum, ileum), large intestine (colon, cecum, appendix, rectum), liver, pancreas, gallbladder, anus, kidney, urinary duct, bladder, prostate, penis, testis, uterus, ovary, vulva, vagina, skin, striated muscle, smooth muscle, synovial membrane, cartilage, bone, thyroid, adrenal gland, peritoneum, mesentery, bone marrow, blood, vascular system, lymphatic system such as lymph node, and lymphatic fluid.

In one embodiment, the cancer in which the RAS/RAF/MEK/ERK signaling cascade is activated may be colon cancer having an activating mutation in KRAS.

As described below, the cell growth inhibitor according to the present invention may be used as a medicament for treating or preventing cancer or used as a research reagent. The cell growth inhibitor according to the present invention may be used in vivo or in vitro. As used herein, the term “in vivo” indicates use for an individual organism, and the term “in vitro” indicates use for tissues or cells isolated from an individual organism.

The present invention also relates to a method of treating or preventing a cancer in which the RAS/RAF/MEK/ERK signaling cascade is activated, the method including using the cell growth inhibitor according to the present invention to inhibit both the GSTP1 autocrine loop and the RAS/RAF/MEK/ERK signaling cascade.

Use of the cell growth inhibitor according to the present invention in treating or preventing cancer will be described later in the section “3. Composition and Treating/Preventing Method”.

The present invention also relates to a method of inhibiting cell growth using the cell growth inhibitor according to the present invention described above. The method may be a method of inhibiting cancer cell growth in vivo, including administering the cell growth inhibitor to a subject or may be a method of inhibiting cancer cell growth in vitro, including administering the cell growth inhibitor to isolated cells or tissues.

In the present disclosure, the inhibition of cell growth may be assessed by a variety of known methods such as counting the number of living cells over time, measuring the size, volume, or weight of a tumor, measuring the amount of synthesized DNA, WST-1 method, BrdU (bromodeoxyuridine) method, and 3H thymidine incorporation assay.

In the present disclosure, examples of cells to be subjected to the in vitro cell growth inhibiting method include, but are not limited to, cancer cells in which the RAS/RAF/MEK/ERK signaling cascade is activated, preferably cancer cells having an activating mutation in a component(s) of the RAS/RAF/MEK/ERK signaling cascade, more preferably cancer cells having an activating mutation in RAS (e.g., KRAS), such as M7609 cells, DLD-1 cells, or HCT116 cells.

It will be understood that those skilled in the art can determine the in vitro dosage as appropriate. For example, the dosage may be such that a medium has an inhibitor concentration of 0.00001 nM to 100000 μM, 0.01 nM to 100 μM, or 1 nM to 1 μM.

The present invention also provides a cell growth inhibitor against a cancer in which the RAS/RAF/MEK/ERK signaling cascade is activated, the cell growth inhibitor including a GSTP1-inhibiting drug to be administered in combination with a RAS/RAF/MEK/ERK signaling cascade-inhibiting drug.

The present invention also provides a cell growth inhibitor against a cancer in which the RAS/RAF/MEK/ERK signaling cascade is activated, the cell growth inhibitor including a RAS/RAF/MEK/ERK signaling cascade-inhibiting drug to be administered in combination with a GSTP1-inhibiting drug.

The GSTP1-inhibiting drug and the RAS/RAF/MEK/ERK signaling cascade-inhibiting drug may be administered simultaneously or at different time points. For administration at different time points, a formulation including the GSTP1-inhibiting drug may be administered before or after a formulation including the RAS/RAF/MEK/ERK signaling cascade-inhibiting drug is administered.

### 2. Cell Growth Inhibitor Including GSTP1-CRAF Interaction-Inhibiting Drug

The present invention also relates to a cell growth inhibitor including a drug that inhibits interaction between GSTP1 and CRAF. The present invention is based on the inventor's findings that cancer cell growth can be inhibited by inhibiting interaction between GSTP1 and CRAF at the junction between the RAS/RAF/MEK/ERK signaling cascade and the GSTP1 autocrine loop shown as Examples described below.

Examples of the “drug that inhibits interaction between GSTP1 and CRAF” include a decoy peptide containing a binding domain (a domain that binds to GSTP1 on CRAF or to CRAF on GSTP1) and having no activity, and a vector that expresses such a decoy peptide. In the present invention, the decoy peptide can competitively inhibit the interaction between endogenous GSTP1 and CRAF. In the present disclosure, a decoy peptide containing a domain that binds to GSTP1 on CRAF and having no CRAF activity is referred to as a CRAF decoy peptide. In the present disclosure, a decoy peptide containing a domain that binds to CRAF on GSTP1 and having no GSTP1 activity is referred to as a GSTP1 decoy peptide.

The drug that inhibits interaction between GSTP1 and CRAF may be a CRAF decoy peptide or a vector that expresses the CRAF decoy peptide.

The CRAF decoy peptide may be selected from the group consisting of

(a) a polypeptide having the amino acid sequence set forth in SEQ ID NO:9,
(b) a polypeptide having an amino acid deletion, substitution, or addition at one or multiple positions in the amino acid sequence set forth in SEQ ID NO: 9,
(c) a polypeptide having at least 90%, 95%, 97%, 98%, or 99% sequence identity to the amino acid sequence set forth in SEQ ID No: 9, and
(d) a polypeptide having 1 to 50 amino acid residues (e.g., 1 to 30, 1 to 20, 1 to 10, or 1 to 5 amino acid residues) added to the N- or C-terminus of the polypeptide defined in any one of (a) to (c).

The amino acid sequence set forth in SEQ ID NO: 9 is the amino acid sequence of positions 56 to 184 of the human CRAF set forth in SEQ ID NO: 5.

Whether the interaction between GSTP1 and CRAF is inhibited may be assessed by detecting the interaction between GSTP1 and CRAF using a known technique such as immunoprecipitation when the drug that inhibits the interaction between GSTP1 and CRAF is allowed to act and when the drug is not allowed to act.

The cell growth inhibitor according to the present invention may be used against a cancer in which the RAS/RAF/MEK/ERK signaling cascade is activated. The cancer in which the RAS/RAF/MEK/ERK signaling cascade is activated is as described above. In one embodiment, the cancer in which the RAS/RAF/MEK/ERK signaling cascade is activated may be a cancer having an activating mutation in RAS (e.g., KRAS). In one embodiment, the cancer in which the RAS/RAF/MEK/ERK signaling cascade is activated may be a cancer that overexpresses GSTP1. In one embodiment, the cancer in which the RAS/RAF/MEK/ERK signaling cascade is activated may be colon cancer having an activating mutation in KRAS.

As described below, the cell growth inhibitor according to the present invention may be used as a medicament for treating or preventing cancer or used as a research reagent. The cell growth inhibitor according to the present invention may be used in vivo or in vitro.

Use of the cell growth inhibitor according to the present invention in treating or preventing cancer is described later in the section “3. Composition and Treating/Preventing Method”.

The present invention also relates to a method of inhibiting cell growth using the cell growth inhibitor according to the present invention described above. The method may be a method of inhibiting cancer cell growth in vivo, including administering the cell growth inhibitor to a subject or a method of inhibiting cancer cell growth in vitro, including administering the cell growth inhibitor to isolated cells or tissues.

It will be understood that those skilled in the art can determine the in vitro dosage as appropriate. For example, the dosage may be such that a medium has an inhibitor concentration of 0.00001 nM to 100000 μM, 0.01 nM to 100 μM, or 1 nM to 1 μM.

The drug that inhibits interaction between GSTP1 and CRAF may be used in combination with at least one of the RAS/RAF/MEK/ERK signaling cascade-inhibiting drug and the GSTP1-inhibiting drug.

### 3. Composition and Treating/Preventing Method

The present invention also relates to a composition including the cell growth inhibitor according to the present invention described above. The composition may be a pharmaceutical composition.

In addition to the active ingredient, the composition may include any other optional ingredient that does not hinder the effect of the active ingredient. Examples of such an optional ingredient include a chemotherapeutic agent and a pharmaceutically acceptable carrier, excipient, or diluent, and so on. Depending on administration route, drug release form, or the like, the pharmaceutical composition may be coated with a suitable material such as an enteric coating or a material that disintegrates over time, or may be incorporated into a suitable drug release system.

The cell growth inhibitor or composition according to the present invention may be administered via various routes including both oral and parenteral routes, examples of which include, but are not limited to, oral, intravenous, intramuscular, subcutaneous, local, intratumoral, rectal, intraarterial, intraportal, intraventricular, transmucosal, transdermal, intranasal, intraperitoneal, intrapulmonary, and intrauterine routes, and may be formulated into a dosage form suitable for each administration route. Any known dosage form and formulation method may be used as appropriate.

Examples of the dosage form suitable for oral administration include, but are not limited to, a powder, granules, a tablet, a capsule, a liquid, a suspension, an emulsion, a gel, and a syrup. Examples of the dosage form suitable for parenteral administration include, but are not limited to, an injection such as a solution injection, a suspension injection, an emulsion injection, or an injection in a form that is prepared at the time of use. A formulation for parenteral administration may be in the form of an aqueous or nonaqueous isotonic sterile solution or suspension.

The cell growth inhibitor or composition according to the present invention may have such an active ingredient content that a desired effect (e.g., a cancer cell growth-inhibiting effect) can be achieved when the cell growth inhibitor or composition is administered. The content is also preferably such that an adverse effect is not produced in such a way as to overwhelm the benefit of the administration. Such a content is known or may be determined as appropriate by in vitro tests using cultured cells or the like or by tests using model animals such as mice, rats, dogs, or pigs, and such test methods are well known to those skilled in the art. The content of the active ingredient may vary depending on the dosage form of the cell growth inhibitor or composition. It will be understood that those skilled in the art can adjust the content as appropriate.

The active ingredient may also be supported on a variety of non-viral lipid or protein carriers. Such carriers include, but are not limited to, cholesterol, liposomes, antibody protomers, cyclodextrin nanoparticles, fusion peptides, aptamers, biodegradable polylactic acid copolymers, and polymers, which can enhance the efficiency of incorporation into cells (see, for example, Pirollo and Chang, Cancer Res. 2008; 68(5): 1247-50). In particular, cationic liposomes or polymers (e.g., polyethyleneimine) are useful. Further examples of polymers useful as such carriers include those disclosed in, for example, US Patent Application, Publication Nos. 2008/0207553 and 2008/0312174.

The cell growth inhibitor or composition according to the present invention may be targeted to a specific tissue or cell. Targeting can be accomplished by any known technique. When delivery to cancer is intended, non-limiting examples of targeting techniques include passive targeting by forming the formulation into diameter sizes of 50 to 200 μm, particularly 75 to 150 μm, which are advantageous for the production of EPR (enhanced permeability and retention) effects; ligands such as CD19, HER2, transferrin receptors, folic acid receptors, VIP receptors, EGFR (Torchilin, AAPS J. 2007; 9(2): E128-47), RAAG10 (JP-T-2005-532050), PIPA (JP-T-2006-506071), or KID3 (JP-T-2007-529197): RGD motif- or NGR motif-containing peptides; and active targeting using F3, LyP-1 (Ruoslahti et al., J Cell Biol. 2010; 188(6): 759-68), or other targeting agents. Since retinoids are known to be useful as cancer cell targeting agents (WO 2008/120815), carriers containing a retinoid as a targeting agent may also be used. Such carriers are disclosed in WO 2009/036368 and WO 2010/014117 as well as in the above documents.

The cell growth inhibitor or composition according to the present invention may be supplied in any form. For storage stability, the cell growth inhibitor or composition according to the present invention may be supplied in a form that can be prepared at the time of use, such as a form that can be prepared by a doctor and/or pharmacist, a nurse, or any other paramedic at or near a medical site. Such a form is particularly useful when the cell growth inhibitor or composition of the present invention contains a component that is difficult to store in a stable condition, such as a lipid, a protein, or a nucleic acid. In this case, the cell growth inhibitor or composition according to the present invention may be prepared, at most 24 hours, preferably at most 3 hours, more preferably immediately before use, from components including at least one essential component, which are provided in one or at least two contains. The preparation may be performed using reagents, solvents, preparation tools, and other materials usually available at the place of preparation.

The specific dose of the cell growth inhibitor or composition according to the present invention may be determined taking into account various conditions related to the subject in need of treatment, such as the degree of severity of conditions, the general health state of the subject, age, body weight, the gender of the subject, diet, the timing and frequency of administration, concomitant pharmaceuticals, the responsiveness to the treatment, the dosage form, and compliance with the treatment. For example, the active ingredient may be administered in an amount of 0.0000001 mg/kg body weight/day to 1,000 mg/kg body weight/day or 0.0001 mg/kg body weight/day to 1 mg/kg body weight/day.

The frequency of administration depends on the properties of the cell growth inhibitor or composition used and the conditions of the subject, which include those mentioned above. For example, the frequency of administration may be multiple times a day (specifically, two, three, four, five, or more times a day), once a day, every few days (specifically, every two, three, four, five, six, seven days, etc.), every week, every few weeks (specifically, every two, three, four weeks, etc.), etc.

The cell growth inhibitor or composition according to the present invention may be used in combination with any other anti-cancer agent. For combination use, it may be a combination drug to be administered simultaneously or may be a separate formulation to be administered independently. The term “combination use” includes simultaneous administration and continuous administration.

The present invention also relates to a method of treating or preventing cancer, comprising administering, to a subject, the cell growth inhibitor or composition according to the present invention described above.

As used herein, the term “treating” includes killing cancer cells, reducing the number of cancer cells, and inhibiting cancer growth. As used herein, the term “preventing” includes prevention of cancer metastasis, prevention of cancer recurrence, and prevention of cancer development.

As used herein, the term “subject” means any individual organism, preferably an animal, more preferably a mammal, even more preferably a human individual. Typically, the subject may be a subject in need of administration of the cell growth inhibitor according to the present invention, such as a subject suffering from cancer, a subject at risk of cancer metastasis or recurrence, or a subject at risk of cancer development.

### 4. Kit

The present invention also relates to a kit for preparing a composition, for inhibiting cell growth, or for treating or preventing cancer, the kit including: the cell growth inhibitor or composition according to the present invention, or an active ingredient or ingredients for forming the cell growth inhibitor or composition; and one or at least two containers containing, singly or in combination, the cell growth inhibitor or composition or an active ingredient or ingredients for forming the cell growth inhibitor or composition.

In addition to the above, the kit of the present invention may include instructions such as written instructions or an electronic recording medium such as a CD or DVD, which shows how to prepare and administer the cell growth inhibitor or composition.

### EXAMPLES

Hereinafter, the present invention will be described in more detail with reference to examples. It will be understood that the examples are not intended to limit the technical scope of the present invention.

### Example 1

### (Analysis of CRAF Domain Binding to GSTP1)

### (1) Cell Culture

The KRAS mutation-positive colon cancer cell line M7609 was cultured in an RPMI medium (containing 10% FBS) at 37° C. The M7609 cells were provided by the Fourth Department of Medicine of Sapporo Medical University. Since M7609 cells have an activating mutation in KRAS, the RAS/RAF/MEK/ERK signaling cascade is activated in M7609 cells.

### (2) Preparation of Plasmids

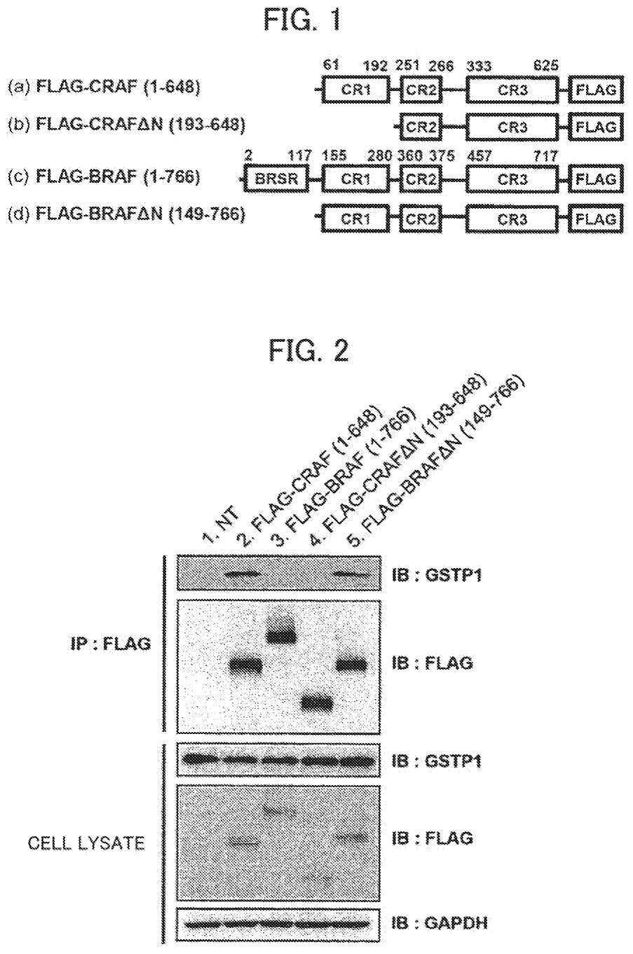

FIG. 1 shows the structures of proteins expressed from plasmids used in Example 1 described below. Part (a) shows FLAG-CRAF (1-648) and the amino acid residue positions of domains in CRAF (positions 61 to 192: CR1 domain, positions 251 to 266: CR2 domain, positions 333 to 625: CR3 domain). Part (b) shows FLAG-CRAFΔN (193-648). Part (c) shows FLAG-BRAF (1-766) and the amino acid residue positions of domains in BRAF (positions 2 to 117: BRSR domain, positions 155 to 280: CR1 domain, positions 360 to 375: CR2 domain, positions 457 to 717: CR3 domain). Part (d) shows FLAG-BRAFΔN (149-766).

FLAG-CRAF (1-648) shown in part (a) of FIG. 1 is a protein having a FLAG tag attached to the C-terminus of the full-length CRAF protein set forth in SEQ ID NO: 5. FLAG-CRAF (1-648) is expressed from the plasmid pcDNA3.1-FLAG-CRAF provided by the Fourth Department of Medicine of Sapporo Medical University.

FLAG-CRAFΔN (193-648) shown in part (b) of FIG. 1 is a protein having a FLAG tag attached to the C-terminus of a deleted form of CRAF protein having the amino acid sequence of positions 193 to 648 of SEQ ID NO: 5. This deleted form of CRAF protein has a deletion at the N-terminal moiety of the full-length CRAF (amino acid residues at positions 1 to 192 of SEQ ID NO: 5). FLAG-CRAFΔN (193-648) is expressed from the plasmid pCMV6-Myc-DDK-CRAFΔN produced by cloning a cDNA corresponding to amino acid residues at positions 193 to 648 of CRAF between AsiSI and MluI sites of a pCMV6-Entry vector (OriGene Technologies).

FLAG-BRAF (1-766) shown in part (c) of FIG. 1 is a protein having a FLAG tag (DDK tag) attached to the C-terminus of the full-length BRAF protein set forth in SEQ ID NO: 10. FLAG-BRAF (1-766) is expressed from the plasmid pCV6-Myc-DDK-BRAF (OriGene Technologies).

FLAG-BRAFΔN (149-766) shown in part (d) of FIG. 1 is a protein having a FLAG tag attached to the C-terminus of a deleted form of BRAF protein having the amino acid sequence of positions 149 to 766 of SEQ ID NO: 10. This deleted form of BRAF protein has a deletion at the N-terminal moiety of the full-length BRAF (amino acid residues at positions 1 to 148 of SEQ ID NO: 10). FLAG-BRAFΔN (149-766) is expressed from the plasmid pCMV6-Myc-DDK-BRAFΔN produced by cloning a cDNA corresponding to amino acid residues at positions 149 to 766 of BRAF between AsiSI and MluI sites of a pCMV6-Entry vector (OriGene Technologies).

### (3) Plasmid Transfection

The M7609 cells were transfected with each of the four plasmids by lipofectamine method. Non-transfected cells (NT) were also prepared as a control.

### (4) Coimmunoprecipitation

The transfected cells were used for coimmunoprecipitation. The cells were lysed by incubation in 0.5% NP-40 lysis buffer (0.5% NP-40, 20 mM HEPES pH 7.4, 150 mM NaCl, 1 mM MgCl₂, 1 mM EGTA, 10% glycerol, Complete, Mini (Roche Diagnostics) and PhosSTOP (Roche Diagnostics)) on ice for 30 min. Centrifugation was performed to give a supernatant as a cell lysate. The cell lysates obtained from the samples and containing the same amount of protein were each incubated with anti-FLAG M2 magnetic beads (Sigma-Aldrich) at 4° C. for 2 h to overnight, so that each FLAG-tagged protein was attached to the beads. The beads were washed four times with 0.5% NP-40 lysis buffer.

### (5) Western Blotting Analysis

Each sample for western blotting was prepared from the beads obtained after the coimmunoprecipitation, and then subjected to SDS-PAGE for protein separation. The separated protein was transferred to a PVDF membrane. The membrane was incubated with a primary antibody solution and then washed. The primary antibody used was an anti-GSTP1 antibody (Medical & Biological Laboratories Co., Ltd.), an anti-FLAG antibody (F3165, Sigma-Aldrich), or an anti-GAPDH antibody (Abcam plc.). The membrane was then incubated with a secondary antibody (goat secondary antibody to rabbit or mouse IgG) solution and then washed. The signal on the membrane was visualized using ECL or ECL prime Western blotting detection system (GE Healthcare). The cell lysates were also subjected to Western blotting analysis in a similar manner.

### (6) Results

FIG. 2 shows the results. GSTP1 was coprecipitated with the full-length CRAF protein (lane 2 in FIG. 2), but not coprecipitated with the CRAF protein with a deletion at the N-terminal moiety (lane 4 in FIG. 2). The results indicate that the N-terminal moiety of the CRAF protein (amino acid residues at positions 1 to 192, including the CR1 domain of amino acid residues at positions 61 to 192) is involved in binding to GSTP1.

In addition, GSTP1 was not coprecipitated with the full-length BRAF protein (lane 3 in FIG. 2), but coprecipitated with the BRAF protein with a deletion at the N-terminal moiety (lane 5 in FIG. 2). The full-length BRAF protein has a high level of amino acid conservation for the full-length CRAF protein, while it has an extension on the N-terminal side (part (c) of FIG. 1). The results of this example show that BRAF with the extension does not bind to GSTP1 and BRAF without the extension binds to GSTP1. This indicates that the presence of the N-terminal extension of BRAF, which is not in CRAF, may sterically interfere with or block binding of GSTP1 to BRAF.

### Example 2

### (Effect of GSTP1 on CRAF Activity)

### (1) Cell Culture and EGF Treatment

KRAS wild-type HeLa cells were cultured in a DMEM medium in an environment of 5% CO₂ at 37° C. The HeLa cells were provided by the Fourth Department of Medicine of Sapporo Medical University. The HeLa cells were first incubated under serum-starved conditions for 16 h and then treated with a 50 ng/ml EGF (epidermal growth factor, BD Biosciences) solution for 10 min. RAS is activated by EGF treatment. RAS-activation is necessary for CRAF phosphorylation.

### (2) In Vitro Kinase Assay

The HeLa cells (with or without EGF-treatment) were transfected with pcDNA3.1-FLAG-CRAF (provided by the Fourth Department of Medicine of Sapporo Medical University) using FuGENE6 HD (Promega Corporation). At 48 h after the transfection, the HeLa cells were lysed by incubation in 0.5% NP-40 lysis buffer (0.5% NP-40, 20 mM HEPES pH 7.4, 150 mM NaCl, 1 mM MgCl₂, 1 mM EGTA, 10% glycerol, Complete, Mini (Roche Diagnostics) and PhosSTOP (Roche Diagnostics)) on ice for 30 min. Centrifugation at 13,000×g for 10 min at 4° C. was performed to give a supernatant as a cell lysate. The cell lysate was incubated with anti-FLAG M2 affinity gel (Sigma-Aldrich) at 4° C. for 2 h so that FLAG-CRAF was bound to the anti-FLAG M2 affinity gel. After the incubation, the anti-FLAG M2 affinity gel was washed four times with 0.5% NP-40 lysis buffer. The FLAG-CRAF-bound anti-FLAG M2 affinity gel was washed with Assay Dilution Buffer I (Merck-Millipore). The gel was then incubated with 1 μg of inactive MEK1 (Merck-Millipore) and magnesium/ATP cocktail (Merck-Millipore) in Assay Dilution Buffer I (Merck-Millipore) at 30° C. for 1 h in the presence or absence of 1 μg of human placental GSTP1 (Sigma-Aldrich) so that MEK1 was phosphorylated by FLAG-CRAF. In addition, a control sample was prepared by incubating active CRAF (OriGene Technologies), instead of the FLAG-CRAF-bound anti-FLAG M2 affinity gel, with inert MEK1 and magnesium/ATP cocktail in Assay Dilution Buffer I.

### (3) Western Blotting Analysis

The samples obtained after the reaction in the in vitro kinase assay was subjected to western blotting analysis as shown in (5) of Example 1, except that the primary antibody used was an anti-p-MEK1/2 (Ser 217/221) antibody (Cell Signaling Technology Inc.) or an anti-MEK1/2 antibody (Cell Signaling Technology Inc.).

### (4) Results

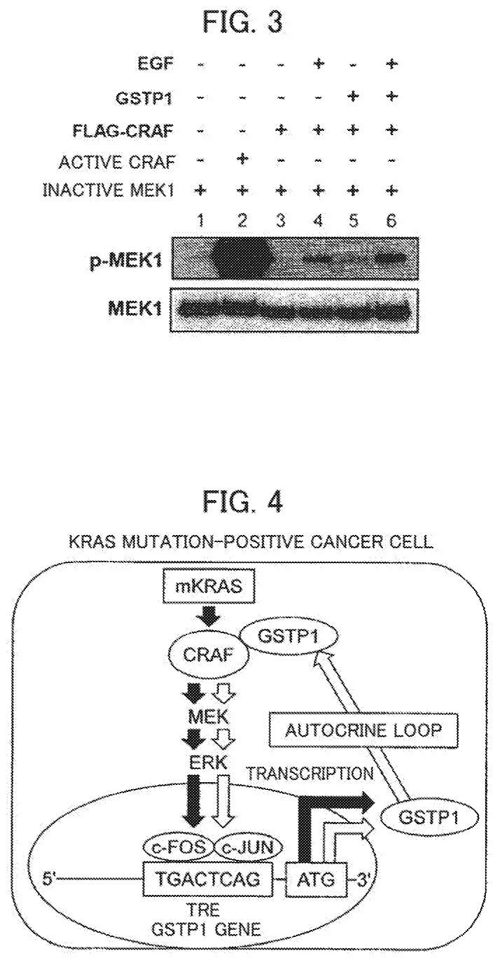

FIG. 3 shows the results. GSTP1 was found to enhance the phosphorylation of MEK1 by FLAG-CRAF (compare lanes 3 and 5 in FIG. 3). In particular, the enhancement of MEK1 phosphorylation by GSTP1 was more evident when FLAG-CRAF isolated from EGF-treated cells was used (compare lanes 4 and 6 in FIG. 3). These results indicate that GSTP1 enhances the activity of CRAF.

It is known that, in cancers in which the RAS/RAF/MEK/ERK signaling cascade is activated, the activation of the signaling cascade may increase the expression of the downstream GSTP1 (the black arrow in FIG. 4). For example, in KRAS mutation-positive cancer cells, constitutively activated variant KRAS (mKRAS) drives signal transduction to RAF such as CRAF, MEK, and ERK, resulting in abnormal cell growth, and the driven signaling cascade induces GSTP1 transcription via binding to phorbol 12-O-tetradecanoate-13-acetate (TPA)-responsive element (TRE; base sequence TGACTCAG) by transcription factors, such as c-FOS and c-JUN (the black arrow in FIG. 4). The results of Examples 1 and 2 indicate that, once GSTP1 is induced by the activation of the RAS/RAF/MEK/ERK signaling cascade (the black arrow pathway in FIG. 4), it enhances the activity of CRAF, a component of the signaling cascade, to drive this cascade independently of the constitutive upstream stimulus by the mutant KRAS (GSTP1 autocrine loop: the white arrow pathway in FIG. 4), so that the signaling cascade is aberrantly activated by both pathways to permanently promote cell growth. It is concluded that inhibition of both pathways is effective in inhibiting cell growth.

### Example 3

### (Effect of CRAF Protein Fragment on Cell Growth)

The CRAF protein fragment expression plasmid pcDNA-hRAF384 (5807 bp) was prepared by cloning the region of positions 332 to 718 of the human CRAF gene (SEQ ID NO: 6) downstream of a CMV (cytomegalovirus) promoter in the vector pcDNA3.1(+) (Thermo Fisher Scientific). The plasmid expresses a CRAF protein fragment (a polypeptide having the amino acid sequence set forth in SEQ ID NO: 9) of positions 56 to 184 of the human CRAF protein (SEQ ID NO: 5).

HCT116 cells (KRAS mutation-positive human colon cancer cells) were obtained from ATCC. Since HCT116 cells have KRAS in which the position 13 amino acid glycine (G) is activatingly mutated to aspartic acid (D), the RAS/RAF/ME/ERK signaling cascade is activated in HCT116 cells.

DLD-1 cells (KRAS mutation-positive human colon cancer cells) were obtained from JCRB Cell Bank of National Institutes of Biomedical innovation, Health and Nutrition. Since DLD-1 cells have KRAS in which the position 13 amino acid glycine (G) is activatingly mutated to aspartic acid (D), the RAS/RAF/MEK/ERK signaling cascade is activated in DLD-1 cells.

The HCT116 or DLD-1 cells were seeded at 1.0×10⁴ or 2.0 x 10⁴ cells per well of 96-well plates and cultured in a McCoy's SA medium in an environment of 5% CO₂ at 37° C. for 24 h. The cultured cells were transfected with the CRAF protein fragment expression plasmid pcDNA-hRAF384 using Lipofectamine 3000 (Thermo Fisher Scientific). As a control, the cells were transfected with the empty vector pcDNA3.1(+) (Thermo Fisher Scientific). The transfection was performed according to the manufacturer's protocol using 0.2 μl of Lipofectamine 3000 and 100 ng of the plasmid per well.

Immediately (0 h), 24 h, 48 h, 72 h, and 96 h after the cultivation, the cells were collected and measured for the number of living cells by WST assay. The WST assay involved using Cell Counting Kit-8 (CCK-8, Dojindo Laboratories) and preforming incubation for 1 h after addition of the CCK-8 solution.

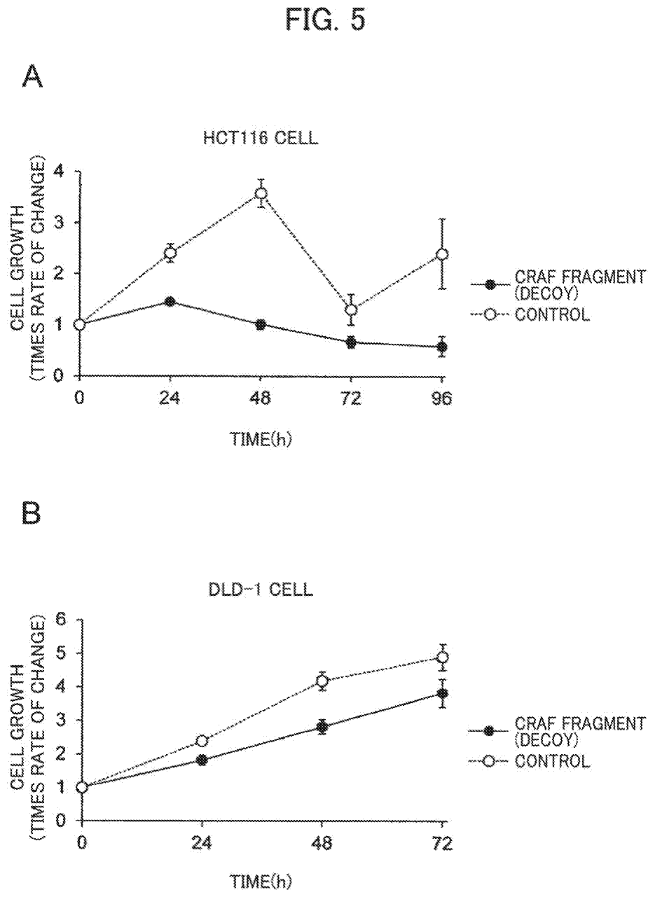

The results obtained when 1.0×10⁴ cells were seeded are shown in part A (HCT116 cells) and part B (DLD-1 cells) of FIG. 5. The cells expressing the CRAF protein fragment showed reduced growth as compared to the control. A similar tendency was also observed when 2.0×10⁴ cells were seeded. The CRAF protein fragment was found to competitively inhibit binding between endogenous GSTP1 and CRAF (i.e., to function as a decoy peptide), which results in blockage of the GSTP1 autocrine loop shown in FIG. 4, inhibition of the RAS/RAF/MEK/ERK cascade-induced cell growth signaling, and inhibition of cell growth.

### Example 4

### (Effect of Double Inhibition of GSTP1 and KARS on Cell Growth)

The KRAS mutation-positive colon cancer cell line M7609 was transfected twice with siRNA. Each transfection was performed using Lipofectamine RNAiMAX (Thermo Fisher Scientific) according to the manufacturer's protocol. The M7609 cells were cultured in an RPMI-1640 medium (antibiotic-free) at 37° C., and after reaching 20 to 30% confluence, the cells were incubated with siRNA in Opti-MEM I (Thermo Fisher Scientific) for 5 h for first transfection. After the transfection, the cells were cultured in an RPMI-1640 medium (antibiotic-free) at 37° C. After culturing for 2 days, second transfection was performed in a similar manner to the first one. After the transfection, the cells were cultured in an RPMI-1640 medium (antibiotic-free) at 37° C. After culturing for 3 days, the total number of the cells was counted. As a control, the number of non-transfected cells (NT) was also counted in a similar manner. The experiment was performed three times independently, and the mean and standard deviation were calculated.

The first and second transfections were performed using the following siRNA.

TABLE 1

| | First | Second |
|---|---|---|
| siControl | Control siRNA | Control siRNA |
| siKRAS | Control siRNA | KRAS siRNA |
| SiGSTP1 | GSTP1 siRNA | Control siRNA |
| siGSTP1 + siKRAS | GSTP1 siRNA | KRAS siRNA |

GSTP1 siRNA was obtained with siRNA ID: 2385 from Thermo Fisher Scientific. The transfection concentration of GSTP1 siRNA was 50 nM. KRAS siRNA was obtained with siRNA ID: s7939 from Thermo Fisher Scientific. The transfection concentration of KRAS siRNA was 10 nM. AllStars Negative Control siRNA (Qiagen) was used as a control siRNA.

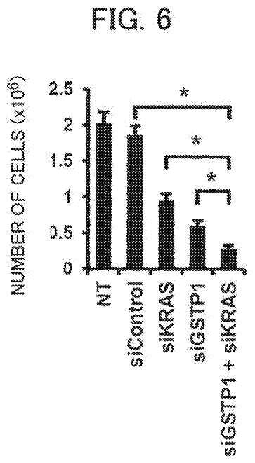

FIG. 6 shows the results. It was found that, while GSTP1 siRNA and KRAS siRNA each independently inhibited KRAS mutation-positive cancer cell growth, the use of a combination of GSTP1 siRNA and KRAS siRNA considerably enhanced the cell growth inhibition.

When KRAS alone is inhibited, signal transduction from the mutant KRAS shown in FIG. 4 is inhibited, but signal transduction by the GSTP1 autocrine loop is not inhibited. When GSTP1 alone is inhibited, the signal transduction by the GSTP1 autocrine loop shown in FIG. 4 is inhibited, but signal transduction from the mutant KRAS is not inhibited. On the other hand, the inhibition of both KRAS and GSTP1 was found to be more effective in inhibiting the RAS/RAF/MEK/ERK signaling cascade and inhibiting cell growth.

All publications, patents, and patent applications cited herein are incorporated herein by reference in their entirety.

1. A cell growth inhibitor against a cancer in which a RAS/RAF/MEK/ERK signaling cascade is activated, the cell growth inhibitor comprising a combination of a GSTP1-inhibiting drug and a RAS/RAF/MEK/ERK signaling cascade-inhibiting drug.

2. A cell growth inhibitor against a cancer in which a RAS/RAF/MEK/ERK signaling cascade is activated, the cell growth inhibitor comprising a GSTP1-inhibiting drug to be administered in combination with a RAS/RAF/MEK/ERK signaling cascade-inhibiting drug.

3. A cell growth inhibitor against a cancer in which a RAS/RAF/MEK/ERK signaling cascade is activated, the cell growth inhibitor comprising a RAS/RAF/MEK/ERK signaling cascade-inhibiting drug to be administered in combination with a GSTP1-inhibiting drug.

4. The cell growth inhibitor according to claim 1, wherein the cancer in which a RAS/RAF/MEK/ERK signaling cascade is activated is a cancer having an activating mutation in RAS.

5. The cell growth inhibitor according to claim 1, wherein the cancer is colon cancer.

6. The cell growth inhibitor according to claim 1, wherein the RAS/RAF/MEK/ERK signaling cascade-inhibiting drug is a RAS-inhibiting drug.

7. The cell growth inhibitor according to claim 1, wherein the GSTP1-inhibiting drug is a siRNA against GSTP1.

8. The cell growth inhibitor according to claim 1, wherein the RAS/RAF/MEK/ERK signaling cascade-inhibiting drug is a siRNA against a component of the RAS/RAF/MEK/ERK signaling cascade.

9. A cell growth inhibitor against a cancer in which a RAS/RAF/MEK/ERK signaling cascade is activated, the cell growth inhibitor comprising a drug that inhibits interaction between GSTP1 and CRAF.

10. The cell growth inhibitor according to claim 9, wherein the cancer in which a RAS/RAF/MEK/ERK signaling cascade is activated is a cancer having an activating mutation in RAS.

11. The cell growth inhibitor according to claim 9, wherein the cancer is colon cancer.

12. The cell growth inhibitor according to claim 9, wherein the drug that inhibits interaction between GSTP1 and CRAF is a CRAF decoy peptide or a vector that expresses the CRAF decoy peptide.

13. The cell growth inhibitor according to claim 12, wherein the CRAF decoy peptide is selected from the group consisting of
(a) a polypeptide having an amino acid sequence set forth in SEQ ID NO:9,
(b) a polypeptide having an amino acid deletion, substitution, or addition at one or multiple positions in the amino acid sequence set forth in SEQ ID NO: 9,
(c) a polypeptide having at least 90% sequence identity to the amino acid sequence set forth in SEQ ID No: 9, and
(d) a polypeptide having 1 to 50 amino acid residues added to an N- or C-terminus of the polypeptide defined in any one of (a) to (c).

14. A pharmaceutical composition for treating or preventing a cancer in which a RAS/RAF/MEK/ERK signaling cascade is activated, the pharmaceutical composition comprising the cell growth inhibitor according to claim 1.

15. A kit for treating or preventing a cancer in which a RAS/RAF/MEK/ERK signaling cascade is activated, the kit comprising the cell growth inhibitor according to claim 1.


SEQUENCE LISTING (10 sequences)

SEQ ID NO: 1 - protein, 210 aa, Homo sapiens
MPPYTVVYFPVRGRCAALRMLLADQGQSWKEEVVTVETWQEGSLKASCLYGQLPKFQDGDLTLYQSNTILRHLGRTLGLYGKDQQEAALVDMVNDGVEDLRCKYISLIYTNYEAGKDDYVKALPGQLKPFETLLSQNQGGKTFIVGDQISFADYNLLDLLLIHEVLAPGCLDAFPLLSAYVGRLSARPKLKAFLASPEYVNLPINGNGKQ

SEQ ID NO: 2 - DNA, 986 bp, Homo sapiens
  CDS (250)..(882)
tgggaaagagggaaaggcttccccggccagctgcgcggcgactccggggactccagggcgcccctctgcggccgacgcccggggtgcagcggccgccggggctggggccggcgggagtccgcgggaccctccagaagagcggccggcgccgtgactcagcactggggcggagcggggcgggaccacccttataaggctcggaggccgcgaggccttcgctggagtttcgccgccgcagtcttcgccaccatgccgccctacaccgtggtctatttcccagttcgaggccgctgcgcggccctgcgcatgctgctggcagatcagggccagagctggaaggaggaggtggtgaccgtggagacgtggcaggagggctcactcaaagcctcctgcctatacgggcagctccccaagttccaggacggagacctcaccctgtaccagtccaataccatcctgcgtcacctgggccgcacccttgggctctatgggaaggaccagcaggaggcagccctggtggacatggtgaatgacggcgtggaggacctccgctgcaaatacatctccctcatctacaccaactatgaggcgggcaaggatgactatgtgaaggcactgcccgggcaactgaagccttttgagaccctgctgtcccagaaccagggaggcaagaccttcattgtgggagaccagatctccttcgctgactacaacctgctggacttgctgctgatccatgaggtcctagcccctggctgcctggatgcgttccccctgctctcagcatatgtggggcgcctcagtgcccggcccaagctcaaggccttcctggcctcccctgagtacgtgaacctccccatcaatggcaacgggaaacagtgagggttggggggactctgagcgggaggcagagtttgccttcctttctccaggaccaataaaatttctaagagagctaaaaaaaaaaaaaaaaaaaaaaaaaaaaa

SEQ ID NO: 3 - protein, 189 aa, Homo sapiens [sequence omitted]

SEQ ID NO: 4 - DNA, 5889 bp, Homo sapiens
  CDS (193)..(762)
tcctaggcggcggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagagagtggaggatgctttttatacattggtgagggagatccgacaatacagattgaaaaaaatcagcaaagaagaaaagactcctggctgtgtgaaaattaaaaaatgcattataatgtaatctgggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggtttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccgcacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgactatgagtgtgtatttattcatgaaatttgaactgtttgccccgaaatggatatggaatactttataagccatagacactatagtataccagtgaatcttttatgcagcttgttagaagtatcctttatttctaaaaggtgctgtggatattatgtaaaggcgtgtttgcttaaacttaaaaccatatttagaagtagatgcaaaacaaatctgcctttatgacaaaaaaataggataacattatttatttatttccttttatcaaagaaggtaattgatacacaacaggtgacttggttttaggcccaaaggtagcagcagcaacattaataatggaaataattgaatagttagttatgtatgttaatgccagtcaccagcaggctatttcaaggtcagaagtaatgactccatacatattatttatttctataactacatttaaatcattaccagg

SEQ ID NO: 5 - protein, 648 aa, Homo sapiens [sequence omitted]

SEQ ID NO: 6 - DNA, 3026 bp, Homo sapiens
  CDS (167)..(2113)
agagctgccccatatctgttgtcacttggatgggccaattccttttctcttgggccgccgaatgtgggacccgggcttgcaccctttctcagggtacttcagtcaagtgacacccttttagagacgacgtgaggaatcgggtttaagaattgtttaagctgcatcaatggagcacatacagggagcttggaagacgatcagcaatggttttggattcaaagatgccgtgtttgatggctccagctgcatctctcctacaatagttcagcagtttggctatcagcgccgggcatcagatgatggcaaactcacagatccttctaagacaagcaacactatccgtgttttcttgccgaacaagcaaagaacagtggtcaatgtgcgaaatggaatgagcttgcatgactgccttatgaaagcactcaaggtgaggggcctgcaaccagagtgctgtgcagtgttcagacttctccacgaacacaaaggtaaaaaagcacgcttagattggaatactgatgctgcgtctttgattggagaagaacttcaagtagatttcctggatcatgttcccctcacaacacacaactttgctcggaagacgttcctgaagcttgccttctgtgacatctgtcagaaattcctgctcaatggatttcgatgtcagacttgtggctacaaatttcatgagcactgtagcaccaaagtacctactatgtgtgtggactggagtaacatcagacaactcttattgtttccaaattccactattggtgatagtggagtcccagcactaccttctttgactatgcgtcgtatgcgagagtctgtttccaggatgcctgttagttctcagcacagatattctacacctcacgccttcacctttaacacctccagtccctcatctgaaggttccctctcccagaggcagaggtcgacatccacacctaatgtccacatggtcagcaccaccctgcctgtggacagcaggatgattgaggatgcaattcgaagtcacagcgaatcagcctcaccttcagccctgtccagtagccccaacaatctgagcccaacaggctggtcacagccgaaaacccccgtgccagcacaaagagagcgggcaccagtatctgggacccaggagaaaaacaaaattaggcctcgtggacagagagattcaagctattattgggaaatagaagccagtgaagtgatgctgtccactcggattgggtcaggctcttttggaactgtttataagggtaaatggcacggagatgttgcagtaaagatcctaaaggttgtcgacccaaccccagagcaattccaggccttcaggaatgaggtggctgttctgcgcaaaacacggcatgtgaacattctgcttttcatggggtacatgacaaaggacaacctggcaattgtgacccagtggtgcgagggcagcagcctctacaaacacctgcatgtccaggagaccaagtttcagatgttccagctaattgacattgcccggcagacggctcagggaatggactatttgcatgcaaagaacatcatccatagagacatgaaatccaacaatatatttctccatgaaggcttaacagtgaaaattggagattttggtttggcaacagtaaagtcacgctggagtggttctcagcaggttgaacaacctactggctctgtcctctggatggccccagaggtgatccgaatgcaggataacaacccattcagtttccagtcggatgtctactcctatggcatcgtattgtatgaactgatgacgggggagcttccttattctcacatcaacaaccgagatcagatcatcttcatggtgggccgaggatatgcctccccagatcttagtaagctatataagaactgccccaaagcaatgaagaggctggtagctgactgtgtgaagaaagtaaaggaagagaggcctctttttccccagatcctgtcttccattgagctgctccaacactctctaccgaagatcaaccggagcgcttccgagccatccttgcatcgggcagcccacactgaggatatcaatgcttgcacgctgaccacgtccccgaggctgcctgtcttctagttgactttgcacctgtcttcaggctgccaggggaggaggagaagccagcaggcaccacttttctgctccctttctccagaggcagaacacatgttttcagagaagctgctgctaaggaccttctagactgctcacagggccttaacttcatgttgccttcttttctatccctttgggccctgggagaaggaagccatttgcagtgctggtgtgtcctgctccctccccacattccccatgctcaaggcccagccttctgtagatgcgcaagtggatgttgatggtagtacaaaaagcaggggcccagccccagctgttggctacatgagtatttagaggaagtaaggtagcaggcagtccagccctgatgtggagacacatgggattttggaaatcagcttctggaggaatgcatgtcacaggcgggactttcttcagagagtggtgcagcgccagacattttgcacataaggcaccaaacagcccaggactgccgagactctggccgcccgaaggagcctgctttggtactatggaacttttcttaggggacacgtcctcctttcacagcttctaaggtgtccagtgcattgggatggttttccaggcaaggcactcggccaatccgcatctcagccctctcagggagcagtcttccatcatgctgaattttgtcttccaggagctgcccctatggggcggggccgcagggccagccttgtttctctaacaaacaaacaaacaaacagccttgtttctctagtcacatcatgtgtatacaaggaagccaggaatacaggttttcttgatgatttgggttttaattttgtttttattgcacctgacaaaatacagttatctgatggtccctcaattatgttattttaataaaataaattaaatttaggtgtaa

SEQ ID NO: 7 - protein, 567 aa, Homo sapiens
MMANSQILLRQATLSVFSCRTSKEQCKKARLDWNTDAASLIGEELQVDFLDHVPLTTHNFARKTFLKLAFCDICQKFLLNGFRCQTCGYKFHEHCSTKVPTMCVDWSNIRQLLLFPNSTIGDSGVPALPSLTMRRMRESVSRMPVSSQHRYSTPHAFTFNTSSPSSEGSLSQRQRSTSTPNVHMVSTTLPVDSRMIEDAIRSHSESASPSALSSSPNNLSPTGWSQPKTPVPAQRERAPVSGTQEKNKIRPRGQRDSSYYWEIEASEVMLSTRIGSGSFGTVYKGKWHGDVAVKILKVVDPTPEQFQAFRNEVAVLRKTRHVNILLFMGYMTKDNLAIVTQWCEGSSLYKHLHVQETKFQMFQLIDIARQTAQGMDYLHAKNIIHRDMKSNNIFLHEGLTVKIGDFGLATVKSRWSGSQQVEQPTGSVLWMAPEVIRMQDNNPFSFQSDVYSYGIVLYELMTGELPYSHINNRDQIIFMVGRGYASPDLSKLYKNCPKAMKRLVADCVKKVKEERPLFPQILSSIELLQHSLPKINRSASEPSLHRAAHTEDINACTLTTSPRLPVF

SEQ ID NO: 8 - DNA, 3255 bp, Homo sapiens
  CDS (639)..(2342)
agaatcggagagccggtggcgtcgcaggtcgggaggacgagcaccgagtcgagggctcgctcgtctgggccgcccgagagtcttaatcgcgggcgcttgggccgccatcttagatggcgggagtaagaggaaaacgattgtgaggcgggaacggctttctgctgccttttttgggccccgaaaagggtcagctggccgggctttggggcgcgtgccctgaggcgcggagcgcgtttgctacgatgcgggggctgctcggggctccgtcccctgggctggggacgcgccgaatgtgaccgcctcccgctccctcacccgccgcggggaggaggagcgggcgagaagctgccgccgaacgacaggacgttggggcggcctggctccctcagacggggtcttggtatgtttcccagagctggtgttaaactcctgggctcaagtagtcctcccatctcagccttccagagtcctgggatttgcaggtttaagaattgtttaagctgcatcaatggagcacatacagggagcttggaagacgatcagcaatggttttggattcaaagatgccgtgtttgatggctccagctgcatctctcctacaatagttcagcagtttggctatcagcgccgggcatcagatgatggcaaactcacagatccttctaagacaagcaacactatccgtgttttcttgccgaacaagcaaagaacagtgtaaaaaagcacgcttagattggaatactgatgctgcgtctttgattggagaagaacttcaagtagatttcctggatcatgttcccctcacaacacacaactttgctcggaagacgttcctgaagcttgccttctgtgacatctgtcagaaattcctgctcaatggatttcgatgtcagacttgtggctacaaatttcatgagcactgtagcaccaaagtacctactatgtgtgtggactggagtaacatcagacaactcttattgtttccaaattccactattggtgatagtggagtcccagcactaccttctttgactatgcgtcgtatgcgagagtctgtttccaggatgcctgttagttctcagcacagatattctacacctcacgccttcacctttaacacctccagtccctcatctgaaggttccctctcccagaggcagaggtcgacatccacacctaatgtccacatggtcagcaccaccctgcctgtggacagcaggatgattgaggatgcaattcgaagtcacagcgaatcagcctcaccttcagccctgtccagtagccccaacaatctgagcccaacaggctggtcacagccgaaaacccccgtgccagcacaaagagagcgggcaccagtatctgggacccaggagaaaaacaaaattaggcctcgtggacagagagattcaagctattattgggaaatagaagccagtgaagtgatgctgtccactcggattgggtcaggctcttttggaactgtttataagggtaaatggcacggagatgttgcagtaaagatcctaaaggttgtcgacccaaccccagagcaattccaggccttcaggaatgaggtggctgttctgcgcaaaacacggcatgtgaacattctgcttttcatggggtacatgacaaaggacaacctggcaattgtgacccagtggtgcgagggcagcagcctctacaaacacctgcatgtccaggagaccaagtttcagatgttccagctaattgacattgcccggcagacggctcagggaatggactatttgcatgcaaagaacatcatccatagagacatgaaatccaacaatatatttctccatgaaggcttaacagtgaaaattggagattttggtttggcaacagtaaagtcacgctggagtggttctcagcaggttgaacaacctactggctctgtcctctggatggccccagaggtgatccgaatgcaggataacaacccattcagtttccagtcggatgtctactcctatggcatcgtattgtatgaactgatgacgggggagcttccttattctcacatcaacaaccgagatcagatcatcttcatggtgggccgaggatatgcctccccagatcttagtaagctatataagaactgccccaaagcaatgaagaggctggtagctgactgtgtgaagaaagtaaaggaagagaggcctctttttccccagatcctgtcttccattgagctgctccaacactctctaccgaagatcaaccggagcgcttccgagccatccttgcatcgggcagcccacactgaggatatcaatgcttgcacgctgaccacgtccccgaggctgcctgtcttctagttgactttgcacctgtcttcaggctgccaggggaggaggagaagccagcaggcaccacttttctgctccctttctccagaggcagaacacatgttttcagagaagctgctgctaaggaccttctagactgctcacagggccttaacttcatgttgccttcttttctatccctttgggccctgggagaaggaagccatttgcagtgctggtgtgtcctgctccctccccacattccccatgctcaaggcccagccttctgtagatgcgcaagtggatgttgatggtagtacaaaaagcaggggcccagccccagctgttggctacatgagtatttagaggaagtaaggtagcaggcagtccagccctgatgtggagacacatgggattttggaaatcagcttctggaggaatgcatgtcacaggcgggactttcttcagagagtggtgcagcgccagacattttgcacataaggcaccaaacagcccaggactgccgagactctggccgcccgaaggagcctgctttggtactatggaacttttcttaggggacacgtcctcctttcacagcttctaaggtgtccagtgcattgggatggttttccaggcaaggcactcggccaatccgcatctcagccctctcagggagcagtcttccatcatgctgaattttgtcttccaggagctgcccctatggggcggggccgcagggccagccttgtttctctaacaaacaaacaaacaaacagccttgtttctctagtcacatcatgtgtatacaaggaagccaggaatacaggttttcttgatgatttgggttttaattttgtttttattgcacctgacaaaatacagttatctgatggtccctcaattatgttattttaataaaataaattaaatttaggtgtaa

SEQ ID NO: 9 - protein, 129 aa, Homo sapiens
NTIRVFLPNKQRTVVNVRNGMSLHDCLMKALKVRGLQPECCAVFRLLHEHKGKKARLDWNTDAASLIGEELQVDFLDHVPLTTHNFARKTFLKLAFCDICQKFLLNGFRCQTCGYKFHEHCSTKVPTMC

SEQ ID NO: 10 - protein, 766 aa, Homo sapiens [sequence omitted]